(19) United States
(12) Patent Application Publication
(10) Pub. No.: US 2015/0246010 A1
(43) Pub. Date: Sep. 3, 2015

(54) HISTONE ACETYL TRANSFERASE ACTIVATORS AND HISTONE DEACETYLASE INHIBITORS IN THE TREATMENT OF ALCOHOLISM

(71) Applicant: THE BOARD OF TRUSTEES OF THE UNIVERSITY OF ILLINOIS, Urbana, IL (US)

(72) Inventor: Subhash C. Pandey, Chicago, IL (US)

(73) Assignees: THE BOARD OF TRUSTEES OF THE UNIVERSITY OF ILLINOIS, Urbana, IL (US); The United States Government as represented by the Department of Veterans Affairs, Washington, DC (US)

(21) Appl. No.: 14/698,412

(22) Filed: Apr. 28, 2015

Related U.S. Application Data
(63) Continuation-in-part of application No. 12/442,908, filed on Jan. 8, 2010.
(60) Provisional application No. 60/848,237, filed on Sep. 29, 2006.

(51) Int. Cl. A61K 31/185; A61K 49/00

(52) U.S. Cl. CPC A61K 31/185; A61K 49/0008

(57) ABSTRACT

The present invention relates to the reduction of a symptom of an alcohol withdrawal state comprising administering a modulator of histone acetylation.

### CROSS-REFERENCE TO RELATED APPLICATIONS

This application is a continuation-in-part of U.S. application Ser. No. 12/442,908, filed Mar. 25, 2009, which is the U.S. national phase of PCT/US07/79944, filed Sep. 28, 2007, which in turn claims priority of U.S. provisional patent application Ser. No. 60/848,237, filed Sep. 29, 2006, the entire respective disclosures of which are incorporated herein by reference.

### GOVERNMENT RIGHTS

This invention was made with government support under National Institutes of Health National Institute on Alcohol Abuse and Alcoholism grant No. R01 AA016690, No. R01 AA010005, No. R01 AA013341, and No. R21 AA015626, and under the Department of Veterans Affairs Merit Review Grant, and Research Career Scientist Award. The government has certain rights to the invention.

### FIELD OF THE INVENTION

The present invention relates generally to the fields of medicine, cellular biology and enzyme biochemistry. More particularly, the invention relates to methods to alleviate symptoms of alcohol withdrawal syndrome using modulators of histone acetylation.

### BACKGROUND OF THE INVENTION

Alcohol withdrawal syndrome frequently ensues following cessation of chronic ethanol consumption and has a significant negative impact on the success of alcoholism treatment regimens. Symptoms indicative of an alcohol withdrawal state include anxiety, fear, muscular rigidity, seizure, and autonomic hyperactivity such as quickened pulse and sweating. Additional symptoms diagnostic of alcohol withdrawal include tremor, insomnia, nausea, vomiting, psychomotor agitation, and transient visual, tactile, or auditory hallucinations. Anxiety commonly occurs as an early symptom of ethanol withdrawal. Onset of this symptom typically begins within 6 to 12 hours after cessation of alcohol use, and as a result, anxiety provides a significant stimulus for the continued use of alcohol by alcoholics. Alcohol produces anxiolytic effects, prompting continued consumption of alcohol to avoid the subsequent occurrence of physical signs of withdrawal. The limbic structures of the brain are important centers for anxiety, and human and rat studies have shown that activation of the various nuclei of amygdala results in anxiety. In particular, the central nucleus of the amygdala (CeA) has been found to be an important regulator of anxiety related to alcohol withdrawal and motivational aspects of alcohol drinking behaviors in rats.

Chronic ethanol exposure is associated with various alterations in cyclic adenosine monophosphate (cAMP)- and Ca²⁺-inducible signaling cascades. In particular, gene transcription patterns of the cAMP-responsive element binding protein (CREB) gene transcription factor are regulated by cAMP and Ca²⁺ (Silva et al, Annu. Rev. Neurosci. 21:127-148, 1998; Montiminy, Annu. Rev. Biochem. 66:808-822, 1997). CREB is a nuclear protein, and is activated by phosphorylation at serine-133, a process carried out by protein kinases including cAMP dependent protein kinase A (PKA), Ca²⁺/calmodulin-dependent protein kinases, and mitogen-activated protein kinases (Meyer et al, Endocrine Reviews 14:269-290, 1993; Hagiwara et al, Mol. Cell. Biol. 13:4852-4859, 1993; Soderling, Trends Biochem. Sci. 24:232-236, 1999; Impey et al, Neuron 23:11-14, 1999). After dimerization, phosphorylated CREB (pCREB) modulates the expression of various cAMP-inducible genes (Montiminy, Annu. Rev. Biochem. 66:808-822, 1997; Xu et al, Neuron 20:709-726, 1998). Several studies in rat brain demonstrate that chronic ethanol treatment decreases the activity of adenylyl cyclase (required for cAMP production), decreases the expression of the stimulatory G protein of adenylyl cyclase (Gs), and increases the expression and function of inhibitory G protein (Gi) (Hoffman et al, Fed. Am. Soc. Exp. Biol. J. 4:2612-2622, 1990; Wand et al, Alcohol Clin. Exp. Res. 15:705-710, 1991; Wand et al, J. Biol. Chem. 268:2595-2601, 1991). PKA-mediated phosphorylation also is decreased in the chronic ethanol-treated rat brain compared with normal control brain (Ruis et al, Brain Res. 365:355-359, 1988), and activation of cAMP-dependent PKA reverses the tolerance of a nucleotide transporter to ethanol (Coe et al, J. Pharmacol. Exp. Ther. 276:365-369, 1996). Chronic ethanol treatment produces a significant reduction in the protein level of regulatory subunits of PKA, and a significant translocation of the catalytic subunits of PKA from the area of the Golgi to the nucleus in NG108-15 cells (Dohrman et al, Proc. Natl. Acad. Sci. USA 93:10217-10221, 1996). Decreased cAMP response element (CRE)-DNA binding activity and decreased phosphorylation of CREB in the rat striatum and in the granule cells of the cerebellum are also associated with chronic ethanol exposure (Yang et al, J. Neurochem. 70:224-232, 1998; Yang et al, Alcohol Clin. Exp. Res. 22:382-390, 1998). Furthermore, using genetic and pharmacological manipulations in Drosophila, decreased function of the cAMP signal transduction pathway was found to be involved in behavioral responses to ethanol intoxication (Moore et al, Cell 93:997-1007, 1998). Taken together, these results suggest that various steps in the cAMP signal transduction cascade are altered in the rat brain and in other cell systems during chronic ethanol exposure.

Although these studies indicate that neuroadaptive changes occur in cAMP-dependent secondary messenger systems during chronic ethanol exposure, they do not clarify how changes in the cAMP-signaling pathway lead to the behavioral symptoms of ethanol withdrawal. Early alcohol withdrawal symptoms such as anxiety play a pivotal role in the continued consumption of alcohol by alcoholics (Roelofs, Alcohol 2:501-505, 1985; Kushner et al, Am. J. Psychiatry 147:685-695, 1990; Weiss et al, J. Clin. Psychiatry 46:3-4, 1985; Schuckit et al, Am. J. Psychiatry 151:1723-1734, 1994), and in the development of alcohol dependence and relapse. Rats subjected to 24 hours of ethanol withdrawal after chronic ethanol treatment develop anxiogenic behaviors, and CREB phosphorylation and Ca²⁺/calmodulin-dependent protein kinase IV expression are significantly decreased in the neurocircuitry of cortical and amygdaloid brain structures (Pandey et al, J. Pharmaco. Exp. Ther. 296:857-868, 2001; Pandey et al, J. Pharmacol. Exp. Ther. 288:866-878, 1999; Pandey et al, Alcohol Clin. Exp. Res. 27:396-409, 2003.). Infusion of PKA activator, but not PKA inhibitor, into the CeA during ethanol withdrawal significantly normalizes the decrease in CREB phosphorylation and also blocks ethanol withdrawal-related anxiety. Thus, decreased CREB phosphorylation in the CeA is associated with anxiety during ethanol withdrawal (Pandey et al. Alcohol Clin. Exp. Res. 27:396-409, 2003).

CREB regulates expression of neuropeptide Y (NPY), a highly abundant peptide that functions as a potent endogenous anxiolytic compound. Chronic ethanol consumption followed by ethanol withdrawal in rats produces significantly lower mRNA and protein levels of NPY in the CeA and medial nucleus of the amygdala (MeA) (Zhang et al, Peptides 24:1397-1402, 2003). Therefore, increased anxiety during ethanol withdrawal may stem from decreased NPY expression due to the decrease in pCREB. In contrast to the effects of ethanol withdrawal, acute ethanol intake produces anxiolytic effects and increased pCREB and NPY levels in the CeA and MeA of mice and alcohol-preferring rats (Pandey et al, J. Neuroscience 24:5022-5030, 2004; Pandey et al, J. Clinical Investigation 115:2762-2773, 2005.). Thus, therapeutic strategies aimed at increasing the level of CREB-inducible genes such as NPY may similarly produce anxiolytic effects.

In addition to gene transcription factors such as CREB, regulation of gene expression requires the recruitment of multifunctional coactivators such as CREB binding protein (CBP) and p300 (Rosenfeld et al, J. Biol. Chem. 276: 36865-36868, 2001; Chrivia et al, Nature 365: 855-859, 1993; Ogryzko et al, Cell 87:953-959, 1996). CBP displays histone acetyltransferase (HAT) activity and the HAT activity of CBP contributes to chromatin remodeling and the resulting modulation of gene expression (Ogryzko et al, Cell 87:953-959, 1996). Chromatin structure plays a key role in mediating changes in gene expression during synaptic plasticity (Hsieh et al, Curr. Op. Cell. Biol. 17:664-671, 2005; Levenson et al, Nat. Rev. Neurosci. 6:108-118, 2005). The fundamental unit of chromatin, a complex of DNA, histones, and non-histone proteins, is the nucleosome. Each nucleosome consists of approximately 147 base pairs of DNA wrapped 1.65 turns around a histone octamer core. The histone core is composed of a central heterotetramer of histones H3 and H4, flanked by two heterodimers of histones H2A and H2B. Several epigenetic mechanisms regulate gene transcription by modulating the accessibility of DNA to the transcriptional machinery. Such mechanisms include DNA methylation and histone acetylation, methylation, and phosphorylation (Hsieh et al, Curr. Op. Cell. Biol. 17:664-671, 2005; Levenson et al, Nat. Rev. Neurosci. 6:108-118, 2005; van Steensel et al, Nat. Genet. (suppl) 37:518-524, 2005; Verdone et al, Biochem. Cell. Biol. 83:344-353, 2005; Colvis et al, J. Neurosci. 25:10379-10389, 2005; Egger et al, Nature 429:457-463, 2004). Acetylation at lysine residues on the N-terminal tails of histones is modulated by histone acetyltransferases (HATs) and histone deacetylases (HDACs). HATs increase histone acetylation resulting in decreased binding to DNA, increased relaxation of nucleosomes, and increased gene expression (Hsieh et al, Curr. Op. Cell. Biol. 17:664-671, 2005). In contract, HDACs reduce histone acetylation resulting in packaging of DNA, more condensed chromatin, and decreased gene expression (Hsieh et al, Curr. Op. Cell. Biol. 17:664-671, 2005; Turner Cell 111:285-291, 2002).

U.S. Patent Publication No. 2006/0018921 relates to the enhancement of cognition by a histone acetylation regulator, such as a histone deacetylase inhibitor. In a specific aspect, the disclosure is directed to enhancing memory that may comprise substantially normal memory faculty or substantially sub-normal memory faculty. In specific embodiments, the sub-normal memory faculty results from a pathogenic condition, such as alcoholism.

U.S. Patent Publication No. 2004/0142859 relates to treatment of diseases and disorders with a deacetylase inhibitor. In specific aspects, these diseases and disorders include polyglutamine expansion diseases such as Huntington's disease, neurological degeneration, psychiatric disorders, and protein aggregation disorders and diseases. The invention is also directed to a transgenic fly useful as a model of polyglutamine expansion diseases.

U.S. Patent Publication No. 2006/0276393 relates to treatment and prevention of neurodegenerative disorders or blood coagulation disorders with a modulator of a sirtuin. In a specific embodiment, a sirtuin activating compound may be used to treat trauma to the nerves, including environmental trauma such as alcoholism.

Several publications by Barlow et. al. pertain to treatment of diseases and conditions of the central and peripheral nervous system by stimulating or increasing neurogenesis using a histone deacetylate inhibitor (U.S. Patent Publication No. 2007/0078083), a modulator of gamma-aminobutyrate receptor activity (U.S. Patent Publication No. 2007/0112017), and a modulator of muscarinic receptor (U.S. Patent Publication No. 2007/0049576). In a specific embodiment, the nervous system disorder may be related to toxic chemicals (e.g. alcohol). In another embodiment, the nervous system disorder may be a psychiatric condition such as alcohol abuse.

Thus, to improve compliance and outcomes in alcohol dependence treatment regimens, a need exists in the art for a method that reduces symptoms associated with an alcohol withdrawal state.

### SUMMARY OF THE INVENTION

The present invention is directed to a method for reducing symptoms related to alcohol withdrawal. A symptom of an alcohol withdrawal state is reduced by administering a modulator of histone acetylation in an amount effective to reduce the symptom of the alcohol withdrawal state. The invention also relates to a method for reducing a desire to consume alcohol. The desire to consume alcohol is reduced by administering a modulator of histone acetylation in an amount effective to reduce the desire to consume alcohol. The invention is further directed to a method for identifying a pharmaceutical agent to treat a symptom of an alcohol withdrawal state. The pharmaceutical agent is identified by determining reduction of the symptom of the alcohol withdrawal state in an animal model after administration of a modulator of histone acetylation compared to the symptom in the absence of the modulator.

In one aspect, the modulator is an inhibitor of a histone deacetylase (HDAC). In another aspect, the modulator is an inhibitor of a class I HDAC, a class II HDAC, a class III HDAC, a class IV HDAC, or combinations of a class I HDAC, a class II HDAC, a class III HDAC, or a class IV HDAC. In a specific embodiment, the modulator, based on its chemical structure, is described as a short-chain fatty acid, a hydroxamic acid, an electrophilic ketone, an aminobenzamide, or a cyclic peptide. In another specific embodiment, the modulator is apicidin B, apicidin C, aroyl pyrrolyl hydroxyamides and derivatives thereof, azelaic bishydroxamic acid (ABHA), butyrate, chlamydocin, CI-994, depsipeptide, depudecin, diheteropeptin, FK228, FR901228, Helminthsporium carbonum (HC) toxin, MS-27-275 (MS-275), oxamflatin, phenylbutyrate, 3-(4-aroyl-2-pyrrolyl)-N-hydroxy-2-propenamides and derivatives thereof, pyroxamide, scriptaid, sirtinol, suberoylanilide hydroxamic acid (SAHA) and derivatives thereof, trapoxin A, trapoxin B, trichostatin A (TSA), trichostatin B (TSB), trichostatin C (TSC), and valproate. In yet another specific embodiment the modulator is suberoylanilide hydroxamic acid (SAHA).

In another aspect, the modulator is an activator of a histone acetyltransferase (HAT). In a specific embodiment, the modulator is a stimulator of cAMP formation. In another specific embodiment, the modulator is an activator of cAMP-dependent protein kinase A (PKA), Ca²⁺/calmodulin-dependent protein kinases, or mitogen activated protein (MAP) kinases. In still another specific embodiment, the modulator increases activation, DNA-binding affinity, HAT-binding affinity, expression, or combinations thereof, of cAMP-responsive element binding protein (CREB).

In a specific aspect, the symptom of the alcohol withdrawal state is anxiety, fear, muscular rigidity, seizure, autonomic hyperactivity, tremor, insomnia, nausea, vomiting, psychomotor agitation, transient visual hallucinations, transient tactile hallucinations, transient auditory hallucinations, or combinations of the aforementioned symptoms. In a specific embodiment, the symptom of the alcohol withdrawal state is anxiety.

In yet another aspect, the step of administering the modulator is carried out orally, intraperitoneally, subcutaneously, percutaneously (transdermally), intravenously, intramuscularly, intrathecally, and epidurally.

### BRIEF DESCRIPTION OF THE DRAWINGS

FIG. 1A is a graph showing the results of the light/dark box exploration test of anxiety-like behaviors in rats.

FIG. 1B is a graph showing the results of the elevated plus maze (EPM) test of anxiety-like behaviors in rats.

FIG. 2 is a graph showing HDAC activity in the amygdala of rats.

FIG. 3 is a graph showing histone H3-K9 acetylation in the amygdala of rats.

FIG. 4 is a graph showing HDAC2 expression in the amygdala of rats.

FIG. 5 is a graph showing LIMK1 expression in the amygdala of rats.

### DETAILED DESCRIPTION OF THE INVENTION

The present invention is directed to a method for reducing symptoms related to alcohol withdrawal. The cessation of alcohol use following chronic, and often excessive, alcohol exposure is frequently accompanied by symptoms of alcohol withdrawal, and the occurrence of alcohol withdrawal symptoms often produces sufficient motivation to relapse into alcohol drinking behaviors. In the present invention, a symptom of an alcohol withdrawal state is reduced by administering a modulator of histone acetylation in an amount effective to reduce the symptom. In one aspect, the modulator is an inhibitor of a histone deacetylase (HDAC). In another aspect, the modulator is an activator of a histone acetyltransferase (HAT). CREB binding protein (CBP) is an example of a histone acetyltransferase.

In a specific embodiment, the symptom of the alcohol withdrawal state is a symptom such as anxiety, fear, muscular rigidity, seizure, autonomic hyperactivity, tremor, insomnia, nausea, vomiting, psychomotor agitation, and transient visual, tactile, or auditory hallucinations or illusions. Other symptoms of alcohol withdrawal syndrome are also within the scope of this invention. In a particular embodiment, the symptom of the alcohol withdrawal state is anxiety.

In yet another specific embodiment, the modulator inhibits at least one class I HDAC, class II HDAC, class III HDAC, or class IV HDAC. The modulator may inhibit more than one HDAC, and those HDACs may belong to more than one class, or may belong to the same class.

In another specific embodiment, the modulator, based on its chemical structure, is described as a short-chain fatty acid, a hydroxamic acid, an electrophilic ketone, an aminobenzamide, or a cyclic peptide. The modulator may have a chemical structure other than one that meets these descriptions. In a specific embodiment, the modulator is apicidin B, apicidin C, aroyl pyrrolyl hydroxyamides and derivatives thereof, azelaic bishydroxamic acid (ABHA), butyrate, chlamydocin, CI-994, depsipeptide, depudecin, diheteropeptin, FK228, FR901228, Helminthsporium carbonum (HC) toxin, MS-27-275 (MS-275), oxamflatin, phenylbutyrate, 3-(4-aroyl-2-pyrrolyl)-N-hydroxy-2-propenamides and derivatives thereof, pyroxamide, scriptaid, sirtinol, suberoylanilide hydroxamic acid (SAHA) and derivatives thereof, trapoxin A, trapoxin B, trichostatin A, trichostatin B trichostatin C, or valproate. Other modulators of histone acetylation are also described by this invention. In a particular embodiment, the modulator is trichostatin A. In another embodiment, the modulator is sirtinol. In another embodiment, the modulator is suberoylanilide hydroxamic acid (SAHA).

In a specific embodiment, the modulator is a stimulator of cAMP formation. Adenylyl cyclase is required for cAMP formation, and examples of stimulators of cAMP formation are serotonin, dopamine and norepinephrine. In another specific embodiment, the modulator is an activator of cAMP-dependent protein kinase A (PKA), an activator of Ca²⁺/calmodulin-dependent protein kinases, or an activator of mitogen activated protein (MAP) kinases. In yet another specific embodiment, the modulator increases activation, DNA-binding affinity, HAT-binding affinity, expression, or combinations thereof, of cAMP-responsive element binding protein (CREB).

In another aspect, the step of administering the modulator is carried out orally, intraperitoneally, subcutaneously, percutaneously, intravenously, intramuscularly, intrathecally, and epidurally. Other methods of administering the modulator are also contemplated by this invention. The modulator may be administered to the brain, and may be administered to specific regions of the brain, such as the amygdala.

Administering the modulator, in one aspect, is associated with biochemical changes such as changes in gene expression, protein levels, or enzyme activity which directly or indirectly modulate histone acetylation. For example, increases in expression of cAMP-responsive element binding protein (CREB)-inducible genes in association with administration of the modulator are contemplated. Neuropeptide Y (NPY) and brain-derived neurotrophic factor (BDNF) are examples of CREB-inducible genes that exhibit increased expression when the modulator is administered. In another aspect, administering the modulator is also associated with physical changes such as changes in behavior. Decreased alcohol consumption and decreased anxiety-like behaviors are examples of physical changes that occur in association with administration of the modulator.

The present invention is also directed to a method for reducing a desire to consume alcohol. The desire to consume alcohol is reduced by administering a modulator of histone acetylation in an amount effective to reduce the desire to consume alcohol. Methods for reducing the desire to consume alcohol in an animal model using a modulator of histone acetylation are described in Example 5 of the present disclosure.

The present invention is further directed to a method for identifying a pharmaceutical agent to treat a symptom of an alcohol withdrawal state. The pharmaceutical agent is identified by determining reduction of the symptom of the alcohol withdrawal state in an animal model after administration of a modulator of histone acetylation compared to the symptom in the absence of the modulator.

### Modulation of Histone Acetylation

The present invention provides modulators of histone acetylation for the treatment of symptoms of alcohol withdrawal. Modulation of a biochemical process (e.g. histone acetylation) refers to the down-regulation or up-regulation of the process, or a combination of both down-regulation and up-regulation. In the case of down-regulation, the response is one of inhibition, suppression, or reduction of the process by an experimentally observable amount. Down-regulation of histone acetylation, for example, refers to a measurable reduction in histone acetylation. In the case of up-regulation, the response is one of activation, stimulation, or enhancement of the process by an experimentally observable amount. Up-regulation of histone acetylation, for example, refers to a measurable augmentation of histone acetylation. A modulator refers to the agent or agents administered in carrying out the modulation of the biochemical process. Acceptable modulators are commonly synthetic molecules, natural products, oligonucleotides, peptides, and proteins (including antibodies), but are not limited to these types of compounds, and also include various combinations of agents. Any modulator of histone acetylation that reduces symptoms of alcohol withdrawal is encompassed by this invention.

Histone acetylation levels are typically dictated by the opposing enzymatic activities of histone deacetylases (HDACs) which decrease acetylation and histone acetyltransferases (HATs) which increase acetylation, but modulation of histone acetylation by other mechanisms is within the scope of this invention. Histone acetylation is modulated by directly or indirectly inhibiting HDACs, activating HDACs, inhibiting HATs, activating HATs, and any combination thereof. In one aspect, the invention pertains to modulation of histone acetylation by inhibition of HDACs, and in another aspect, the invention pertains to modulation of histone acetylation by activation of HATs.

The HDAC family is comprised of approximately a dozen enzymes, and the modulator of the present invention, in various aspects, inhibits one HDAC or more than one HDAC, and inhibits HDACs belonging to the same class or HDACs belonging to multiple classes. Class I and class II enzymes are closely related and share a common catalytic mechanism. Examples of class I HDACs include HDAC1, HDAC2, HDAC3, and HDAC8. Class II includes HDAC4, HDAC5, HDAC6, HDAC7, HDAC9 and HDAC10. Class IV HDACs such as HDAC11 are evolutionarily distinct from class I and II (Gallinari et al, Cell Res. 17:195-211, 2007). Class III HDACs, also known as silent information regulator 2 (Sir2) proteins, have a catalytic mechanism differing from that of class I, class II, and class IV HDACs, and require nicotinamide adenine dinucleotide (NAD) as a cofactor.

Numerous HDAC inhibitors are known in the art, many of which have resulted from research efforts directed toward the identification of anti-cancer agents. Trichostatin A (TSA) is one example of an HDAC inhibitor, and TSA potently inhibits class I and class II HDACs (Yoshida et al, J. Biol. Chem. 265:17174-17179, 1990). Sirtinol is a specific inhibitor of Sir-2 (Landry et al, Biochem. Biophys. Res. Comm 278:685-690, 2000; Blander et al, Annu. Rev. Biochem. 73:417-435, 2004). Five classes of HDAC inhibitors are commonly known in the art, and these classes, based on chemical structure of the inhibitors, are short-chain fatty acids, hydroxamic acids, electrophilic ketones, aminobenzamides, and cyclic peptides. Thus, in one aspect, HDAC inhibitors of the present invention belong to any of these classes, but are not limited to these classifications. Specific HDAC inhibitors known in the art and contemplated for use in methods of the invention include apicidin B, apicidin C, aroyl pyrrolyl hydroxyamides and derivatives thereof, azelaic bishydroxamic acid (ABHA), butyrate, chlamydocin, CI-994, depsipeptide, depudecin, diheteropeptin, FK228, FR901228, Helminthsporium carbonum (HC) toxin, MS-27-275 (MS-275), oxamflatin, phenylbutyrate, 3-(4-aroyl-2-pyrrolyl)-N-hydroxy-2-propenamides and derivatives thereof, pyroxamide, scriptaid, sirtinol, suberoylanilide hydroxamic acid (SAHA) and derivatives thereof, trapoxin A, trapoxin B, trichostatin A, trichostatin B trichostatin C, and valproate.

Still other HDAC inhibitors are known in the art, and have been described in publications including AU 9,013,101; AU 9,013,201; AU 9,013,401; AU 6,794,700; EP 1,233,958; EP 1,208,086; EP 1,174,438; EP 1,173,562; EP 1,170,008; EP 1,123,111; JP 2001/348340; U.S. Pat. No. 7,250,514; U.S. Pat. No. 7,199,134; U.S. Pat. No. 7,183,298; U.S. Pat. No. 7,169,801; U.S. Pat. No. 7,154,002; U.S. Pat. No. 7,135,493; U.S. Pat. No. 7,126,001; U.S. Pat. No. 7,098,241; U.S. Pat. No. 7,098,186; U.S. Pat. No. 7,056,883; U.S. Pat. No. 6,960,685; U.S. Pat. No. 6,897,220; U.S. Pat. No. 6,888,027; U.S. Pat. No. 6,800,638; U.S. Pat. No. 6,667,341; U.S. Pat. No. 6,541,661; U.S. Pat. No. 6,531,472; U.S. Pat. No. 6,087,367; U.S. Pat. No. 5,932,616; U.S. Pat. No. 5,840,960; U.S. Pat. No. 5,773,474; U.S. Pat. No. 5,700,811; U.S. Pat. No. 5,668,179; U.S. Pat. No. 5,608,108; U.S. Pat. No. 5,369,108; U.S. Pat. No. 5,330,744; U.S. Pat. No. 5,175,191; U.S. 2002/0103192; U.S. 2002/0061860; WO 02/51842; WO 02/50285; WO 02/46144; WO 02/46129; WO 02/30879; WO 02/26703; WO 02/26696; WO 01/70675; WO 01/42437; WO 01/38322; WO 01/18045; WO 01/14581; Uesato et al, Bioorg. & Med. Chem. Lett. 12:1347-1349, 2002; Finnin et al, Nature 401:188-193, 1999; Richon et al, Proc. Natl. Acad. Sci. 97:10014-10019. 2000; Richon et al, Proc. Natl. Acad. Sci. 95: 3003-3007, 1998; Marks et al, Curr. Opin. Oncol. 13:477-483, 2001; and Kramer et al, Trends Endo. & Metab. 12:294-300, 2001.

Histone acetyltransferases are classified as type A or type B based on the subcellular localization of the enzyme. Type A HATs are located in the nucleus, and many play important roles in the regulation of gene expression by functioning as transcriptional co-activators. Type B HATs are located in the cytoplasm and are intimately involved with chromatin synthesis and assembly of nascent histones into chromosomes. Type A HATs include CBP, p300, Esa1, Gcn5, P/CAF, TAFII250, and Tip60. Members of the p160 family of proteins are also type A HATs and include p/CIP, ACTR, TIF2/GRIP-1/NcoA-2 and SRC-1/NCoA-1. HAT1 is an example of a type B HAT.

Activation of a histone acetyltransferase relates to the stimulation or enhancement of histone acetylation, which occurs directly, i.e. via interaction of a modulator with a HAT, or indirectly as a result of, for example, modulation of upstream signaling events. A small molecule, N-(4-chloro-3-trifluoromethyl-phenyl)-2-ethoxy-6-pentadecyl-benzamide (CTPB) contemplated for use in methods of the invention, is known in the art to activate p300 HAT activity without affecting HDAC activity (Balasubramanyam et al, J. Biol. Chem. 278:19134-19140, 2003). Derivative compounds also contemplated for use in the methods provided, e.g. N-(4-chloro-3-trifluoromethyl-phenyl)-2-ethoxy-benzamide (CTB), are also HAT activators (Mantelingu et al, J. Phys. Chem. B, 111:4527-4534, 2007).

Additional upstream mechanisms exist whereby HATs are activated. For example, stimulation of cAMP formation is a mechanism for activating HATs, as is activation of cAMP-dependent protein kinase A (PKA). The activation of Ca²⁺/calmodulin dependent protein kinases II & IV, and activation of mitogen activated protein (MAP) kinases also are mechanisms for activating HATs. In a specific embodiment of the methods provided, HAT is activated by a modulator that increases activation, DNA-binding affinity, HAT-binding affinity, expression, or combinations thereof, of cAMP-responsive element binding protein (CREB).

The activities of many HATs are regulated through phosphorylation, and in one aspect, a HAT is activated by modulating its phosphorylation. HAT activity of CBP, for example, is stimulated on phosphorylation by cyclin E/cyclin-dependent kinase 2 (Ait-Si-Ali et al, Nature 396:184-186, 1998). HAT activity is modulated via interactions of HATs with specific factors. CBP and p300 are two examples of HATs wherein activity is stimulated in cis by a variety of sequence-specific transcription factors such as HNF1-alpha, HNF4, Sp1, Zta, NF-E2, C/EBP-alpha and phosphorylated Elk1 (Chen et al, Mol. Cell. Biol. 21:476-487, 2001; Li et al, EMBO J. 22:281-291, 2003; Soutoglou et al, EMBO J. 20:1984-1992, 2001). A further mechanism by which HAT activity is modulated is via the availability of cofactors, such as, for example, acetyl-coenzyme A. Still other mechanisms exist whereby a HAT is activated, including for example, through regulation of its stability. Tip60, a HAT that is involved in apoptosis and DNA repair after double-stranded breaks, is an example of a HAT that is degraded by the proteasome after ubiquitin addition by the ubiquitin ligase Mdm2 (Legube et al, EMBO J. 21:1704-1712, 2002). Accordingly, modulators of protein ubiquitylation, e.g. inhibitors of ubiquitin ligases, are also activators of HATs.

### DEFINITIONS

The term “alcohol withdrawal state” means the condition which occurs on cessation or reduction of repeated or chronic alcohol use.

The term “symptom” means a sensation, condition, or sign that accompanies a disease, disorder, or illness.

The term “anxiety” means a state of apprehension or tension.

The term “fear” means a distressing feeling caused by the presence or imminence of danger, whether the threat is real or imagined.

The term “muscular rigidity” refers to an increased resistance of a joint to passive movements.

The term “seizure” refers to a sudden change in behavior due to an excessive electrical activity in the brain. Seizures are “simple”, in which no change in level of consciousness occurs, or “complex”, in which a change in level of consciousness does occur. Seizures that affect the whole body are classified as generalized, and seizures that affect only one part or side of the body are classified as focal.

The term “autonomic hyperactivity” refers to abnormal activity of the autonomic nervous system and includes such non-limiting symptoms as elevated blood pressure, elevated heart rate, dilated pupils, increased sweating, and elevated rate of breathing.

The term “tremor” means involuntary trembling in part of the body.

The term “insomnia” means difficulty in initiating sleep or difficulty in maintaining sleep. Insomnia refers to any and all stages and types of sleep loss.

The term “nausea” means the sensation of having an urge to vomit.

The term “vomiting” means forcing the contents of the stomach up through the esophagus and out of the mouth.

The term “psychomotor agitation” means unintentional motions or purposeless motions that stem from mental tension.

The term “transient visual hallucinations” refers to temporary abnormal sensory perceptions of sight. The term “transient tactile hallucinations” refers to temporary abnormal sensory perceptions of touch. The term “transient auditory hallucinations” refer to temporary abnormal sensory perceptions of hearing.

The term “histone deacetylase”, abbreviated “HDAC”, means all enzymes, including enzymatically active fragments and variants thereof, with measurable activity in catalyzing the deacetylation of histones and includes class I HDACs, class II HDACs, class III HDACs, and class IV HDACs. Class III HDACs are also known as silent information regulator 2 (Sir2) proteins.

The term “histone acetyltransferase”, abbreviated “HAT”, means all enzymes, including enzymatically active fragments and variants thereof, with measurable activity in catalyzing the acetylation of histones and includes CREB binding protein (CBP).

### Dosages, Routes of Administration, and Formulations

An “effective amount,” e.g., dose, of compound or drug for treating a condition described herein is an amount of a therapeutic compound that achieves a desired therapeutic endpoint and is readily be determined by routine experimentation, as can an effective and convenient route of administration and an appropriate formulation. Those of ordinary skill in the art will readily optimize effective dosages and administration regimens as determined by good medical practice and the clinical condition of the individual subject. The frequency of dosing will depend on the pharmacokinetic parameters of the agents and the route of administration. The optimal pharmaceutical formulation will be determined by one skilled in the art depending upon the route of administration and desired dosage. See for example, Remington's Pharmaceutical Sciences, 18th Ed. (1990, Mack Publishing Co., Easton, Pa. 18042, pages 1435-1712), the disclosure of which is hereby incorporated by reference. Such formulations may influence the physical state, stability, rate of in vivo release, and rate of in vivo clearance of the administered agents.

An effective amount of modulator composition will depend, for example, upon the therapeutic context and objectives. The appropriate dosage levels for treatment will thus vary depending, in part, upon the molecule delivered, the symptom for which the modulator is being used, the route of administration, and the size (body weight, body surface or organ size) and condition (the age and general health) of the subject. A typical dosage may range from about 0.1 μg/kg to up to about 100 mg/kg or more, from about 1 μg/kg up to about 100 mg/kg; or 5 μg/kg up to about 100 mg/kg, depending on the factors mentioned above.

Suitable routes of administration may, for example, include oral, rectal, topical, nasal, pulmonary, ocular, intestinal, and parenteral administration. Primary routes for parenteral administration include intravenous, intramuscular, and subcutaneous administration. Additional routes of administration include intraperitoneal, intra-arterial, intra-articular, intracardiac, intracisternal, intradermal, intralesional, intraocular, intrapleural, intrathecal, intrauterine, and intraventricular administration. The severity of the indication to be treated, along with the physical, chemical, and biological properties of the drug, dictate the type of formulation and the route of administration to be used.

Pharmaceutical dosage forms of a compound of the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. The compositions of the present invention can include one or more physiologically acceptable inactive ingredients that facilitate processing of active molecules into preparations for pharmaceutical use.

Pharmaceutical dosage forms of a compound of the invention may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art, and include those listed in various pharmacopoeias. (See, e.g., the U.S. Pharmacopeia (USP), Japanese Pharmacopoeia (JP), European Pharmacopoeia (EP), and British pharmacopeia (BP); the U.S. Food and Drug Administration Center for Drug Evaluation and Research (CEDR) publications, e.g., Inactive Ingredient Guide (1996); Ash and Ash, Eds. (2002) Handbook of Pharmaceutical Additives, Synapse Information Resources, Inc., Endicott N.Y.; etc.)

The following examples are not intended to be limiting but only exemplary of specific embodiments of the invention.

### Example 1

### Effects of Acute Ethanol Exposure on HDAC Activity, Histone Acetylation, and CBP Level in the Amygdala of Rats

The effect of acute ethanol exposure on HDAC activity, histone acetylation level, and CBP level was measured in Sprague-Dawley (SD) rats. Rats were injected with ethanol (1 g/kg intraperitoneal injection) or n-saline, and after one hour, anxiolytic responses were measured. Acute ethanol produced anxiolytic effects in SD rats consistent with the results of similar studies in SD rats, Wistar rats, alcohol-preferring rats and in mice (Pandey et al, J. Neurosci. 24:5022-5030, 2004; Pandey et al, J. Clin. Invest. 115:2762-2773, 2005; Prunell et al, Pharmacol. Biochem. Behav. 47:147-151, 1994; Langen et al, Alcohol 27:135-141, 2002; Pautassi et al, Alcohol Clin. Exp. Res. 30:448-459, 2006; Gallate et al, Psychopharmacology 166:51-60, 2003). The amygdala was then dissected out, and HDAC activity was measured. Acute ethanol inhibited activity of HDACs in the amygdala of Sprague-Dawley (SD) rats by 36% compared to the activity of HDACs in control n-saline-treated rats.

The protein levels of acetylated histone H3, acetylated histone H4, and CBP in the CeA structures of n-saline- or acute ethanol-treated rats were measured by immunohistochemistry. Gold-immunolabeling of acetylated histones H3 and H4 and of CBP in the CeA showed increased protein levels of acetylated H3, acetylated H4, and CBP in acute ethanol-treated rats. Acute ethanol treatment also increased protein levels of acetylated H3, acetylated H4, and CBP in MeA but not in basolateral amygdaloid (BLA) structures of rats.

### Example 2

### Effect of HDAC Inhibitors on Anxiety-Like Behaviors of Ethanol-Withdrawn Rats after Chronic Ethanol Exposure

The effect of HDAC inhibition on anxiety-like behaviors of ethanol-withdrawn SD rats after chronic ethanol exposure was assayed using the class I and class II HDAC inhibitor trichostatin A (TSA). SD rats were fed with control or ethanol liquid diet, and ethanol-fed rats were withdrawn for 24 hours as described previously (Pandey et al, Alcohol Clin. Exp. Res. 27:396-409, 2003). Ethanol-withdrawn and control rats were treated with TSA or vehicle (1:5 dilution of DMSO with phosphate-buffered saline) two hours before measuring anxiety-like behaviors using the elevated-plus maze (EPM) test. The EPM is a cross-shaped elevated apparatus consisting of two open arms and two closed arms arranged directly opposite each other and connected to a central platform. To measure anxiety-like behaviors, a test rat was habituated for five-minutes in the test room and then placed on the central platform facing an open arm. The number of entries to each type of arm over a five-minute period was observed and recorded. EPM test results were reported as the mean±SEM of the percent of open-arm entries and the percent of time spent on the open arms. These collectively are referred to as open-arm activity. The general activity of each rat was measured by calculating the sum of open- and closed-arm entries. In the EPM test, ethanol withdrawal after chronic ethanol exposure produced increased anxiety-like behaviors as measured by a reduction in open-arm activities. TSA treatment restored these anxiety-like behaviors to normal levels. In the light/dark box exploration test of anxiety-like behaviors, ethanol withdrawal after chronic ethanol exposure produced an increase in anxiety-like behaviors as evidenced by reductions in time spent in the light box. As indicated by increased time spent in the light box, treatment with the HDAC inhibitor TSA significantly reduced these anxiety-like behaviors.

### Example 3

### Effects of HDAC Inhibitors on HDAC Activity, Histone Acetylation, CBP, Sir-2, and NPY of Ethanol-Withdrawn Rats after Chronic Ethanol Exposure

The effect on HDAC activity in the amygdala of ethanol-withdrawn SD rats after chronic ethanol exposure was measured. SD rats were fed with control or ethanol liquid diet, and ethanol-fed rats were withdrawn for 24 hours. Ethanol-withdrawn and control rats were treated with TSA or vehicle two hours before measuring HDAC activity. Ethanol withdrawal after chronic ethanol exposure produced an increase in HDAC activity in the amygdala of rats. Treatment of ethanol-withdrawn rats with TSA completely prevented this increase in HDAC activity in the rat amygdala.

The effect of HDAC inhibition on acetylated histone H3, CBP, and Sir-2 (HDAC III) level in ethanol-withdrawn SD rats after chronic ethanol exposure was assayed using trichostatin A (TSA). SD rats were fed control or ethanol liquid diet, and ethanol-fed rats were withdrawn for 24 hours. Ethanol-withdrawn and control rats were treated with TSA or vehicle, and protein levels were measured in amygdaloid structures by gold-immunolabeling. In the CeA and MeA, but not in the BLA, ethanol withdrawal produced significant reductions in protein levels of acetylated histone H3 and CBP-HAT, and produced an increase in protein levels of Sir-2. Treatment of ethanol-withdrawn rats with TSA, an HDAC inhibitor, rescued acetylated histone H3 levels, but did not modulate CBP and Sir-2 levels. Thus, histone acetylation is normalized by TSA treatment due to inhibition of HDAC activity.

The effect of HDAC inhibition on NPY mRNA and protein levels in ethanol-withdrawn SD rats after chronic ethanol exposure was also assayed using trichostatin A (TSA). SD rats were fed control or ethanol liquid diet, and ethanol-fed rats were withdrawn for 24 hours. Ethanol-withdrawn and control rats were treated with TSA or vehicle, and mRNA and protein levels were measured in amygdaloid structures. In the CeA and MeA, but not in the BLA, ethanol withdrawal produced significant reductions in both mRNA and protein levels of NPY. Treatment of ethanol-withdrawn rats with TSA, an HDAC inhibitor, restored NPY levels to normal. Thus, NPY expression is also normalized by TSA treatment during ethanol withdrawal after chronic ethanol exposure.

Therefore, intraperitoneal injection of an HDAC inhibitor such as TSA prevented the development of anxiety-like behaviors, normalized the increase in HDAC activity in the amygdala, normalized the reduction in acetylation of histone H3 in the amygdala, and normalized the reduction in NPY expression in the amygdala in ethanol-withdrawn SD rats after chronic ethanol exposure.

### Example 4

### Effect of Central Amygdaloid Infusion of a Sir-2 (HDAC III) Inhibitor on Anxiety-Like Behaviors of Ethanol-Withdrawn Rats after Chronic Ethanol Exposure

The effect of Sir-2 inhibition on anxiety-like behaviors in ethanol-withdrawn SD rats after chronic ethanol exposure was assayed by central amygdaloid infusion of the Sir-2 inhibitor, sirtinol. SD rats were fed control or ethanol liquid diet, and ethanol-fed rats were withdrawn for 24 hours. Sirtinol (0.5 μl of 25 μM sirtinol) or vehicle (0.5 μl of 0.3% DMSO diluted with artificial CSF) was infused into the CeA of ethanol-withdrawn and control rats, and anxiety was measured after two hours. Anxiety-like behaviors in rats were then measured in the EPM test. Ethanol withdrawal after chronic ethanol exposure produced increased anxiety-like behaviors as evidenced by a reduction in open-arm activities, and development of these anxiety-like behaviors was prevented by central amygdaloid infusion of the Sir-2 inhibitor sirtinol.

### Example 5

### Effect of the HDAC Inhibitor TSA on Alcohol Intake in P and NP Rats

The effect of HDAC inhibition on the alcohol consumption of alcohol-preferring (P) and non-preferring (NP) rats was assayed. Selective breeding has produced the P and NP rat lines with high and low alcohol preference, respectively (Li T. K, Lumeng L, Doolittle D P, Behav. Genet. 23:163-170, 1993; McKinzie D L, Nowak K L, Murphy J M, Li T K, Lumeng L, McBride W J, Alcohol Clin. Exp. Res. 22:1584-1590, 1998). P and NP rats were habituated to drink water equally from two bottles. One bottle was then replaced with 7% ethanol for the first three days and 9% ethanol for the next seven days. During the last three days of 9% alcohol drinking, rats were treated with TSA daily (2 mg/kg intraperitoneal injection). Treatment with TSA significantly attenuated the alcohol intake in high alcohol-drinking P rats, but not in low alcohol-drinking NP rats. As a result of the large reduction in voluntary alcohol consumption by the alcohol-preferring animal model when treated with an HDAC inhibitor, it is expected that HDAC inhibitors also prevent the development of alcohol dependence.

### Example 6

### Effect of the HDAC Inhibitor Suberoylanilide Hydroxamic Acid (SAHA) on Anxiety-Like Behaviors of Ethanol-Withdrawn Rats after Chronic Ethanol Exposure

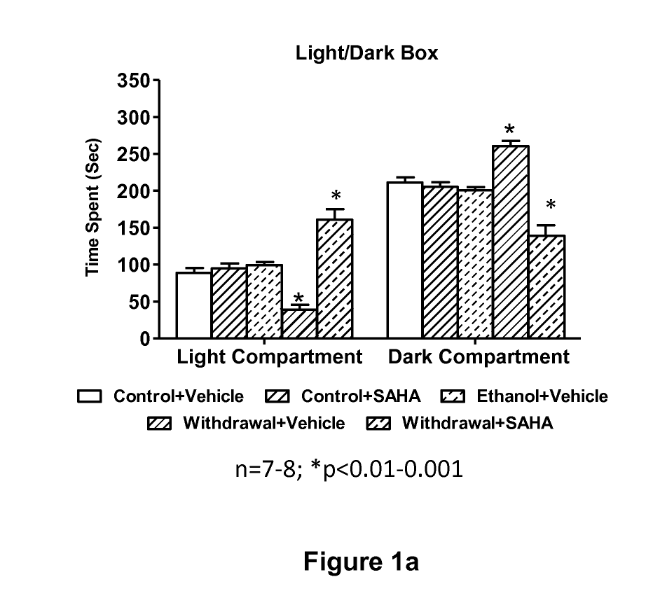

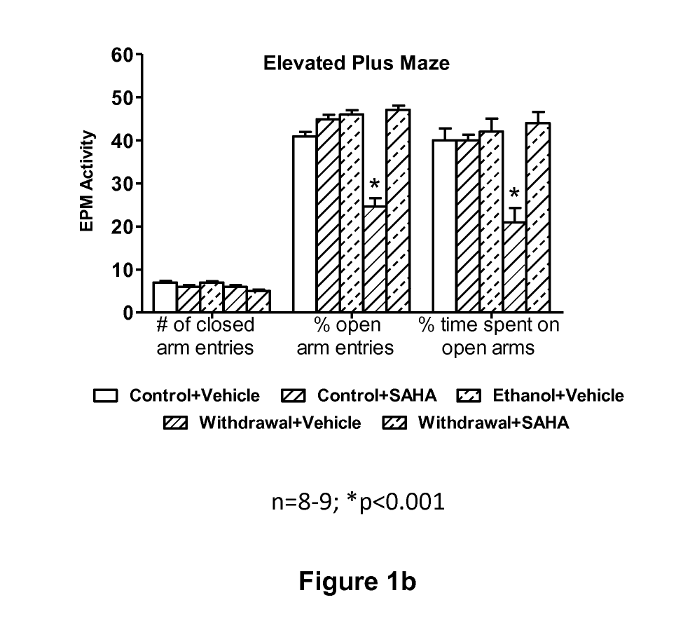

The effect of HDAC inhibition on anxiety-like behaviors of ethanol-withdrawn SD rats after chronic ethanol exposure was assayed using the HDAC inhibitor suberoylanilide hydroxamic acid (SAHA) according to the methods of Example 2, except that SAHA was administered intraperitoneally in an amount of 50 mg/kg in place of TSA. In the light/dark box exploration test of anxiety-like behaviors, ethanol withdrawal after chronic ethanol exposure produced an increase in anxiety-like behaviors as evidenced by reductions in time spent in the light box. As indicated by increased time spent in the light box, treatment with the HDAC inhibitor SAHA significantly reduced these anxiety-like behaviors (FIG. 1A). In the EPM test, ethanol withdrawal after chronic ethanol exposure produced increased anxiety-like behaviors as measured by a reduction in open-arm activities. SAHA treatment prevented these anxiety-like behaviors (FIG. 1B).

### Example 7

### Effect of the HDAC Inhibitor SAHA on HDAC Activity of Ethanol-Withdrawn Rats after Chronic Ethanol Exposure

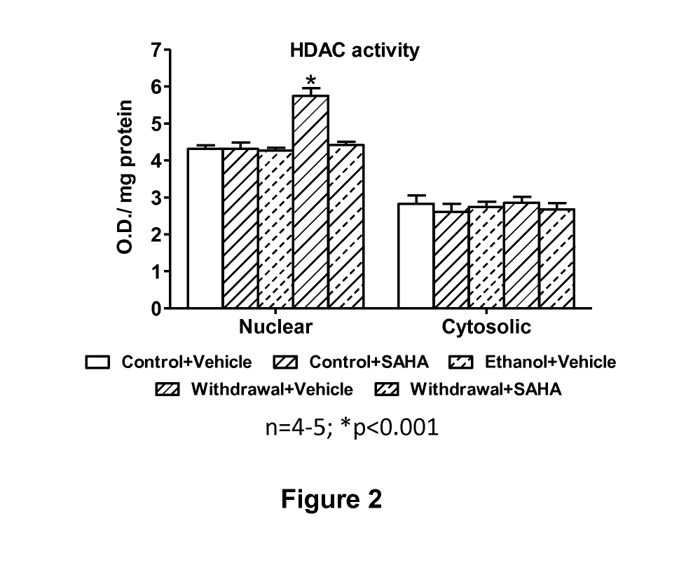

The effect on HDAC activity in the amygdala of ethanol-withdrawn SD rats after chronic ethanol exposure was measured. SD rats were fed with control or ethanol liquid diet, and ethanol-fed rats were withdrawn for 24 hours. Ethanol-withdrawn and control rats were treated with SAHA (intraperitoneally, 50 mg/kg) or vehicle. Two hours after injections, brains were collected to measure HDAC activity. The nuclear and cytosolic fractions of amygdala were prepared and HDAC activities were measured using an HDAC ELISA kit. Ethanol withdrawal after chronic ethanol exposure produced an increase in nuclear, but not cytosolic, HDAC activity in the amygdala of rats. Treatment of ethanol-withdrawn rats with SAHA attenuated this increase in nuclear HDAC activity in the rat amygdala (FIG. 2).

### Example 8

### Effect of the HDAC Inhibitor SAHA on Histone Acetylation of Ethanol-Withdrawn Rats after Chronic Ethanol Exposure

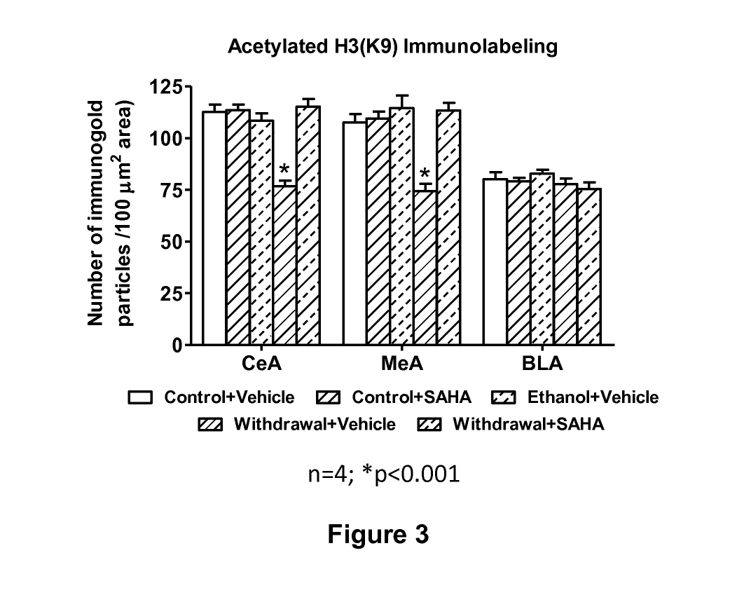

The effect of HDAC inhibition on acetylated histone H3-K9 protein levels in ethanol-withdrawn SD rats after chronic ethanol exposure was assayed using SAHA according to the methods of Example 3, except that SAHA was administered intraperitoneally in an amount of 50 mg/kg in place of TSA. In the CeA and MeA, but not in the BLA, ethanol withdrawal produced significant reductions in protein levels of acetylated histone H3-K9. Treatment of ethanol-withdrawn rats with SAHA corrected the deficits in histone H3-K9 acetylation in the CeA and MeA, but had no effects on histone H3-K9 acetylation in the BLA of ethanol-withdrawn rats (FIG. 3).

### Example 9

### Effect of the HDAC Inhibitor SAHA on HDAC2 Expression in Ethanol-Withdrawn Rats after Chronic Ethanol Exposure

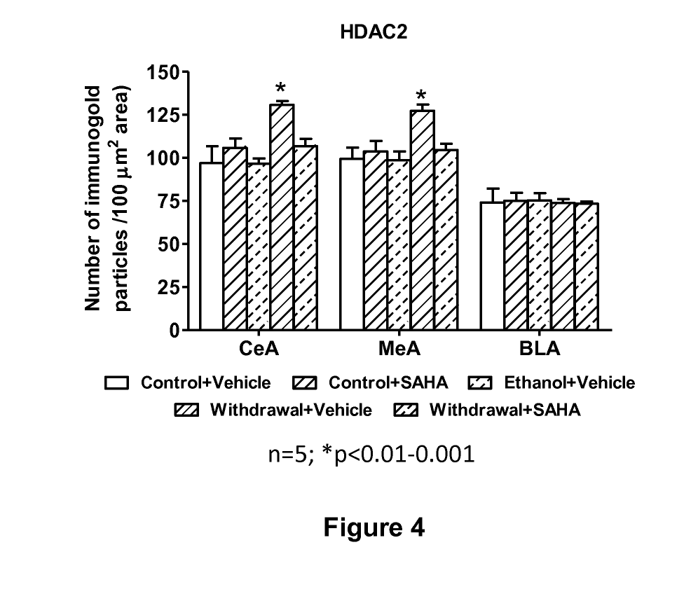

The effect on protein levels of the HDAC2 isoform in the amygdala of ethanol-withdrawn SD rats after chronic ethanol exposure was measured. SD rats were fed with control or ethanol liquid diet, and ethanol-fed rats were withdrawn for 24 hours. Ethanol-withdrawn and control rats were treated with SAHA (intraperitoneally, 50 mg/kg) or vehicle two hours before measuring behaviors. Brains were collected after perfusion for HDAC2 protein level determination. Protein levels were measured in amygdaloid structures by gold-immunolabeling histochemical procedure. In the CeA and MeA, but not in the BLA, ethanol withdrawal produced significant increases in protein levels of the HDAC2 isoform. Treatment with SAHA normalized HDAC2 levels of ethanol-withdrawn rats (FIG. 4).

### Example 10

### Effect of the HDAC Inhibitor SAHA on Limk1 Expression in Ethanol-Withdrawn Rats after Chronic Ethanol Exposure

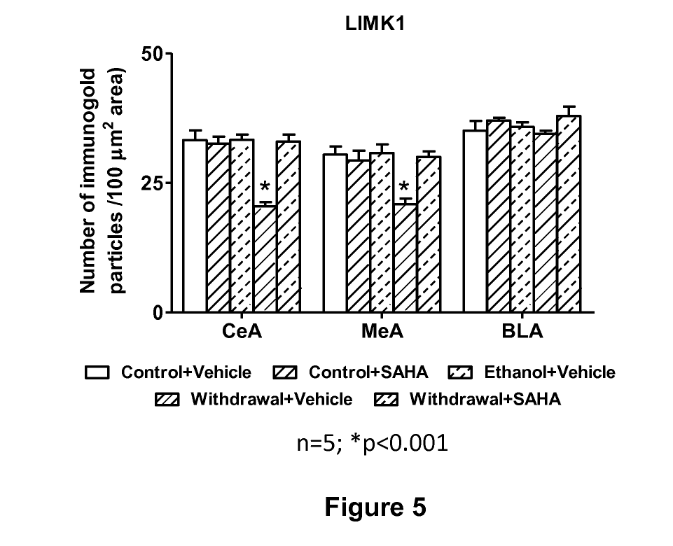

Limk1, a serine protein kinase, has been shown to interact with synaptic plasticity-associated molecules and to be involved in the regulation of dendritic spines. The effect on protein levels of Limk1 in the amygdala of ethanol-withdrawn SD rats after chronic ethanol exposure was measured. SD rats were fed with control or ethanol liquid diet, and ethanol-fed rats were withdrawn for 24 hours. Ethanol-withdrawn and control rats were treated with SAHA (intraperitoneally, 50 mg/kg) or vehicle two hours before measuring behaviors. Brains were collected after perfusion for Limk1 protein level measurement. Protein levels were measured in amygdaloid structures by gold-immunolabeling. Ethanol withdrawal, but not ethanol treatment, produced significant decreases in protein levels of Limk1 in the CeA and MeA, but not in the BLA. Treatment with SAHA normalized Limk1 levels of ethanol-withdrawn rats (FIG. 5). Without wishing to be bound by theory, it is believed that decreased dendritic spine density during ethanol withdrawal could be related to decreased Limk1 protein levels, that Limk1 may be under epigenetic control in the amygdaloid circuitry during ethanol exposure and its withdrawal, and that SAHA is neuro-protective and damage caused by chronic ethanol can be reversed by SAHA treatment.

What is claimed is:

1. A method for reducing a symptom of an alcohol withdrawal state comprising the step of administering a modulator of histone acetylation in an amount effective to reduce the symptom of the alcohol withdrawal state in a subject in an alcohol withdrawal state.

2. The method of claim 1 wherein the modulator is a direct inhibitor of a histone deacetylase (HDAC).

3. The method of claim 1 wherein the modulator is an activator of a histone acetyltransferase (HAT).

4. The method of claim 2 wherein the modulator is an inhibitor of a class I HDAC, a class II HDAC, a class III HDAC, a class IV HDAC, or combinations thereof.

5. The method of claim 2 wherein the modulator is selected from the group consisting of a short-chain fatty acid, a hydroxamic acid, an electrophilic ketone, an aminobenzamide, and a cyclic peptide.

6. The method of claim 2 wherein the modulator is selected from the group consisting of apicidin B, apicidin C, aroyl pyrrolyl hydroxyamides, azelaic bishydroxamic acid (ABHA), butyrate, chlamydocin, CI-994, depsipeptide, depudecin, diheteropeptin, FK228, FR901228, Helminthsporium carbonum (HC) toxin, MS-27-275 (MS-275), oxamflatin, phenylbutyrate, 3-(4-aroyl-2-pyrrolyl)-N-hydroxy-2-propenamides, pyroxamide, scriptaid, sirtinol, suberoylanilide hydroxamic acid (SAHA), trapoxin A, trapoxin B, trichostatin A, trichostatin B, trichostatin C, and valproate.

7. The method of claim 2 wherein the modulator is suberoylanilide hydroxamic acid (SAHA).

8. The method of claim 1 wherein the symptom of the alcohol withdrawal state is selected from the group consisting of anxiety, fear, muscular rigidity, seizure, autonomic hyperactivity, tremor, insomnia, nausea, vomiting, psychomotor agitation, transient visual hallucinations, transient tactile hallucinations, and transient auditory hallucinations.

9. The method of claim 1 wherein the symptom of the alcohol withdrawal state is anxiety.

10. The method of claim 1 wherein the step of administering is carried out orally, intraperitoneally, subcutaneously, percutaneously, intravenously, intramuscularly, intrathecally, and epidurally.

11. A method for reducing a desire to consume alcohol comprising the step of administering a modulator of histone acetylation in an amount effective to reduce the desire to consume alcohol.

12. The method of claim 11 wherein the modulator is a direct inhibitor of a histone deacetylase (HDAC).

13. The method of claim 12 wherein the modulator is selected from the group consisting of apicidin B, apicidin C, aroyl pyrrolyl hydroxyamides, azelaic bishydroxamic acid (ABHA), butyrate, chlamydocin, CI-994, depsipeptide, depudecin, diheteropeptin, FK228, FR901228, Helminthsporium carbonum (HC) toxin, MS-27-275 (MS-275), oxamflatin, phenylbutyrate, 3-(4-aroyl-2-pyrrolyl)-N-hydroxy-2-propenamides, pyroxamide, scriptaid, sirtinol, suberoylanilide hydroxamic acid (SAHA) and derivatives thereof, trapoxin A, trapoxin B, trichostatin A, trichostatin B trichostatin C, and valproate.

14. The method of claim 12 wherein the modulator is suberoylanilide hydroxamic acid (SAHA).

15. The method of claim 11 wherein the step of administering is carried out orally, intraperitoneally, subcutaneously, percutaneously, intravenously, intramuscularly, intrathecally, and epidurally.

16. A method for identifying a pharmaceutical agent to treat a symptom of an alcohol withdrawal state comprising the step determining reduction of the symptom of the alcohol withdrawal state in an animal model after administration of a modulator of histone acetylation compared to the symptom in the absence of the modulator.

17. The method of claim 16 wherein the modulator is a direct inhibitor of a histone deacetylase (HDAC).

18. The method of claim 17 wherein the modulator is selected from the group consisting of apicidin B, apicidin C, aroyl pyrrolyl hydroxyamides, azelaic bishydroxamic acid (ABHA), butyrate, chlamydocin, CI-994, depsipeptide, depudecin, diheteropeptin, FK228, FR901228, Helminthsporium carbonum (HC) toxin, MS-27-275 (MS-275), oxamflatin, phenylbutyrate, 3-(4-aroyl-2-pyrrolyl)-N-hydroxy-2-propenamides, pyroxamide, scriptaid, sirtinol, suberoylanilide hydroxamic acid (SAHA), trapoxin A, trapoxin B, trichostatin A, trichostatin B, trichostatin C, and valproate.

19. The method of claim 17 wherein the modulator is suberoylanilide hydroxamic acid (SAHA).

20. The method of claim 16 wherein the step of administering is carried out orally, intraperitoneally, subcutaneously, percutaneously, intravenously, intramuscularly, intrathecally, and epidurally.
